(19) United States
(12) Patent Application Publication
(10) Pub. No.: US 2005/0119351 A1
(43) Pub. Date: Jun. 2, 2005

(54) Polymorphic forms of sertraline hydrochloride

(76) Inventors: Paul Adriaan Van Der Schaaf, Allschwil (CH); Franz Schwarzenbach, Frenkendorf (CH); Hans-Jorg Kirner, Pratteln (CH); Martin Szelagiewicz, Munchenstein (CH); Claudia Marcolli, Zurich (CH); Andreas Burkhard, Basel (CH)

Correspondence Address: CIBA SPECIALTY CHEMICALS CORPORATION;PATENT DEPARTMENT, 540 WHITE PLAINS RD, P O BOX 2005, TARRYTOWN, NY, 10591-9005, US

(21) Appl. No.: 10/945,786

(22) Filed: Sep. 21, 2004

Related U.S. Application Data
(63) Continuation-in-part of application No. 10/736,195, filed on Dec. 15, 2003.
Application No. 10/736,195 is a continuation of application No. 10/111,947, filed on Apr. 26, 2002, now Pat. No. 6,872,853.

(30) Foreign Application Priority Data
Oct. 29, 1999 (EP) 99810981.3

(51) Int. Cl. A61K 31/135

(52) U.S. Cl. 514/657; 564/428

(57) ABSTRACT

An improved process for the preparation of sertraline hydrochloride polymorphic form II is described, which process comprises seeding a solution of sertraline free amine in a ketone with some crystals of polymorphic form II and addition of hydrogen chloride, and wherein the solution is heated before addition of the hydrogen chloride. According to the present process, the metastable form II may be obtained in a reliable way with good yield and high purity with respect to other polymorphic forms as well as preparative residues such as educts or solvents.

This is a continuation-in-part of application Ser. No. 10/736,195, filed Dec. 15, 2003, which is a continuation of application Ser. No. 10/111,947, filed Apr. 26, 2002, which is the National Stage of International Application No. PCT/EP 00/10416, filed Oct. 23, 2000.

The present invention relates to an improved process for the preparation of crystalline polymorphic form II of (1S-cis)-4-(3,4-dichlorophenyl)-1,2,3,4-tetrahydro-N-methyl-1-naphthalenamine hydrochloride, i.e. sertraline hydrochloride.

Sertraline hydrochloride is useful as an antidepressant and anorectic agent, and is also useful in the treatment of chemical dependencies, anxiety-related disorders and premature ejaculation, and is described in U.S. Pat. No. 4,536,518 (Pfizer Inc.).

Sertraline hydrochloride can exist in different crystalline forms, polymorphic forms, which differ from each other in their stability, physical properties, spectral data and methods of preparation.

Sertraline has the following structural chemical formula:

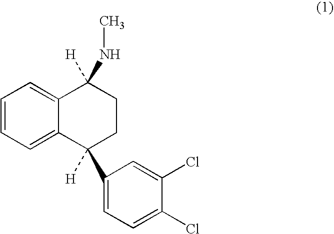

U.S. Pat. No. 4,536,518 (Pfizer Inc.) discloses the synthesis of sertraline hydrochloride. The amine is dissolved in a mixture of diethyl ether and ethyl acetate and reacted with hydrogen chloride gas. The reference states that the compounds of this invention may exist in different polymorphic forms, i.e. different crystalline forms. The reference does not refer to specific polymorphic crystalline forms of sertraline hydrochloride.

U.S. Pat. No. 5,248,699 (Pfizer Inc.) discloses 5 polymorphic forms of sertraline hydrochloride (I, II, III, IV, and V) as well as methods for preparing them. The reference further discloses that the synthetic procedure described and exemplified in U.S. Pat. No. 4,536,518 produces the metastable sertraline hydrochloride Form II.

Although it is disclosed in U.S. Pat. No. 5,248,699 (Pfizer Inc.) that polymorphic form I exhibits the greatest stability of the crystalline forms of sertraline hydrochloride, the solubility of this form may be unsuitable for successful application. For example, the rate of absorption of a drug is dependent upon the dissolution rate. The dissolution rate and the rate of absorption will either increase or decrease depending upon the polymorph present. The most stable polymorph will have the lowest solubility and in many cases the slowest dissolution rate. Other less stable polymorphs will usually have higher dissolution rates. [Stephen R. Byrn in “Solid-State Chemistry of Drugs”, Academic Press, New York, 1982].

Several attempts have been made to improve the process for producing the metastable polymorphic form II of sertraline HCl (see, for example, JP-A-2000-26378; U.S. Pat. No. 6,495,721). The reliability of the known processes and the purity of the product, in terms of crystal form as well as chemical purity, still require improvement.

Sertraline hydrochloride polymorphic form II may be formed from a solution of sertraline free amine with some seeding crystals of form II before or after the addition of hydrogen chloride, e.g. as a solution of hydrogen chloride; or from a stirred suspension of sertraline hydrochloride polymorphic form V with some seeding crystals of sertraline hydrochloride polymorphic form II; or by drying a sertraline hydrochloride alcohol solvate at temperatures from about 0 to 30° C. in high vacuum (<1 mbar); or from stirred suspensions of sertraline hydrochloride polymorphic form CSC1, CSC2 or T1 with some seeding crystals of sertraline hydrochloride polymorphic form II. Sertraline hydrochloride polymorphic form II may further be formed according to a process, wherein a solution of sertraline free amine is seeded with some crystals of polymorphic form II and hydrogen chloride is added (see appl. U.S. Ser. No. 10/111,947; WO01032601).

It has now been found, that the metastable form II may be obtained in a reliable way with good yield and high purity with respect to other polymorphic forms as well as preparative residues such as educts or solvents. The process of the present invention is especially well suitable for industrial use.

In the present improved process for the preparation of sertraline hydrochloride polymorphic form II, a solution of sertraline free amine in a ketone is seeded with some crystals of polymorphic form II and hydrogen chloride is added after heating the solution.

The starting solution in this case is a clear solution not containing any visible traces of crystals of sertraline or sertraline hydrochloride impurities. If desired, the solution may be subjected to an additional filtration step before or after heating. Before addition of seeding crystals and/or hydrogen chloride, the concentration of any form of sertraline dissolved is well below saturation, for example below 80%, preferably below 50% of the saturated solution. For example, ketone solutions containing about 5 to about 20% by weight of sertraline free amine can be used. Any impurity of sertraline hydrochloride at this stage should be avoided; preferably it is, if present, kept below 1% by weight.

In a preferred process, the solution is heated to 45-80° C. prior to addition of hydrogen chloride.

Preferred are ketone solvents of formula R₁—CO—R₂, wherein R₁ and R₂ are C₁-C₄alkyl, especially where R₁ is C₁-C₄alkyl and R₂ is C₂-C₄alkyl. Examples for R₁ and R₂ are methyl, ethyl, n- or i-propyl and n-, s-, i- or t-BUTYL.

R₁ is preferably methyl. R₂ is preferably methyl, ethyl or butyl such as isobutyl. Examples of ketones are acetone, methyl ethyl ketone, methyl propyl ketone, methyl butyl ketone, methyl isobutyl ketone. In a preferred process, a solution of sertraline free amine in a water-immiscible ketone is used.

The hydrogen chloride can be added, for example, as a solution in an organic solvent, like a ketone as given above, as an aqueous solution, or preferably as HCl gas.

In a preferred process, hydrogen chloride is added until a pH less than 5.5, especially less than 5, is reached.

The process is usually carried out with the ketone as the sole solvent, any other solvent, including the one for adding hydrogen chloride, preferably being kept at a level below 20% of the total amount of solvent used. Of special technical interest is a process wherein essentially no other solvent than a ketone is used (and thus present during crystallization).

It is preferred to add the seeding crystals of polymorphic form II before addition of hydrogen chloride. In certain cases, especially if a solution of low concentration is used, however, the order of addition may also be reversed, i.e. hydrogen chloride is added to the solution of sertraline free amine and subsequently the solution is seeded with some crystals of polymorphic form II.

Typical amounts of seeding crystals are 0.1 to 10 mol-%, preferably 1 to 10, especially 2 to 10 mol-%, based on the molar amount of sertraline.

Seeding crystals and HCl or hydrochloric acid usually is mixed with the solution by a separate measure such as stirring.

Workup and isolation of the product, including drying, may be effected using methods known in the art. In a preferred process, sertraline hydrochloride form II is separated from the liquid phase, washed with a pharmaceutically acceptable solvent and dried.

Sertaline free base and its solution in a ketone used in present process may be prepared in situ; thus, present invention further pertains to a process, wherein a solution of sertraline free amine is obtained from sertraline mandelate. In a preferred process, the solution of sertraline free amine is obtained after
a) treating a solution of sertraline mandelate in a ketone immiscible with water, with an aqueous hydroxide,
b) phase separation and
c) washing the ketone solution with water.

This invention also relates to a pharmaceutical composition comprising polymorphic form II as obtained in the present process effective in treating depressions, anxiety-related disorders, obesity, chemical dependencies, or addictions or premature ejaculations in a human, and a pharmaceutically acceptable carrier.

The present polymorphic form may be used as single components or mixtures.

The following examples will illustrate, but do not limit the scope of the present invention. Whereever used, room temperature denotes a temperature in the range 20-30° C. Percentages are given by weight, unless otherwise specified.

### REFERENCE EXAMPLE 1

### Preparation of Polymorphic Form II in Acetone without Heating

24 g sertraline free amine are dissolved in 280 ml acetone. To this solution 1.2 g sertraline hydrochloride polymorphic form II are added as seeding crystals. To this mixture are added dropwise 53.7 g of a solution of HCl in acetone (5.33 wt %) at room temperature. The resulting white suspension is stirred for an additional 2 h, filtered, and the resulting white product washed with acetone (2×20 ml). The product is dried in vacuum (0.1 mbar) for 16 h.

The product is obtained in 91% isolated yield.

Raman and X-ray powder diffraction studies show the product to be polymorphic form II (see FIGS. 3 and 4).

### REFERENCE EXAMPLE 2

### Low Amount of Seeding Crystals, No Heating

95 g sertraline free amine are dissolved in 1100 ml of acetone. To this solution 0.10 g sertraline hydrochloride polymorphic form II are added as seeding crystals. HCl in acetone (5.33 wt %) is subsequently added and workup effected as described in reference example 1. Slightly more than 100 g of sertraline hydrochloride are obtained after drying; X-ray powder diffraction gives a pattern as in FIG. 1, showing that the product entirely consists of sertraline form I hydrochloride.

### REFERENCE EXAMPLE 3

### Hydrochloride Impurity, No Heating

Following the procedure described in reference example 1, a mixture containing 70 mol-% of the sertraline as free amine, 20 mol-% as form II hydrochloride and 10 mol-% as form I hydrochloride are dissolved in acetone at room temperature. To this solution, 5 mol-% of sertraline hydrochloride polymorphic form II are added as seeding crystals. HCl in acetone (5.33 wt %) is subsequently added dropwise over a period of 1 hour at room temperature. The resulting suspension is stirred for an additional 2 h, filtered, and dried in vacuum.

X-ray powder diffraction gives a pattern as in FIG. 1, showing that the product consists of pure sertraline form I hydrochloride.

### EXAMPLE 1

### Preparation of Polymorphic Form II in Acetone Using Aqueous HCl

50 g of sertraline free amine are dissolved in 500 ml acetone. This solution is clarified by filtration, and the clear solution is well stirred and heated to reflux temperature. At this temperature 2.5 g (5 mol %) of sertraline hydrochloride Form II are added after which directly the addition of the aqueous hydrogenchloride solution (32%) is started until pH<5. The resulting white suspension is cooled to −5° C. and sertraline hydrochloride is isolated by filtration and dried in vacuum. The sertraline is obtained as Form II.

### EXAMPLE 2

### Preparation of Polymorphic Form II Using Methyl Ethyl Ketone and ag. HCl

10 g sertraline free amine are dissolved in 85 ml methyl ethyl ketone. This solution is clarified by filtration, and the clear solution is well stirred and heated to 60° C. At this temperature 0.5 g (5 mol %) of sertraline hydrochloride Form II are added after which directly the addition of the aqueous hydrogen chloride solution (37%) is started until pH<5. The resulting white suspension is cooled to −5° C. and sertraline hydrochloride is isolated by filtration and dried in vacuum. The sertraline is obtained as Form II.

### EXAMPLE 3

### Preparation of Polymorphic Form II Using Methyl Isobutyl Ketone and ag. HCl

40 g sertraline free amine are dissolved in 360 ml methyl isobutyl ketone. This solution is clarified by filtration, and the clear solution is well stirred and heated to 60° C. At this temperature 2 g (5 mol %) of sertraline hydrochloride Form II are added after which directly the addition of the aqueous hydrogen chloride solution (37%) is started until pH<5. The resulting white suspension is cooled to 20° C. and sertraline hydrochloride is isolated by filtration and dried in vacuum. The sertraline is obtained as Form II.

### EXAMPLE 4

### Preparation of Polymorphic Form II Using Methyl Isobutyl Ketone and HCl Gas

129.6 kg of enantiopure sertraline mandelate are dissolved in methyl isobutyl ketone. Sodium hydroxide (50% aqueous solution) is added to convert the mandelate into sertraline free base. The organic phase is separated, washed with water, and dried by azeotropic distillation. The organic phase is cooled to room temperature, filtered, and subsequently heated to 55-60° C. Seeding crystals of sertraline HCl form II (4.8 kg) are added, and subsequently HCl gas is bubbled into the liquid until pH 4 is reached, while the mixture is constantly stirred. The mixture is cooled in 2 steps to 40-45° C. and then to 15-20° C., where the solid product is filtered off, washed with methyl isobutyl ketone and subsequently with isopropyl acetate, and then dried at 30° C. 100.4 kg of pure sertraline form II hydrochloride (97.2% relative to mandelate educt) are obtained (identified by X-ray powder diffraction, pattern as in FIG. 3).

### BRIEF DESCRIPTION OF THE DRAWINGS

FIG. 1 is a characteristic X-ray powder diffraction pattern for polymorphic form I

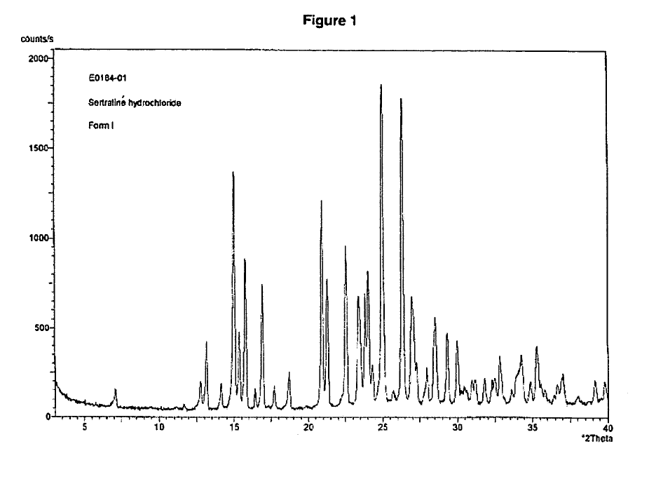

FIG. 2 is a characteristic Raman spectrum of polymorphic form I

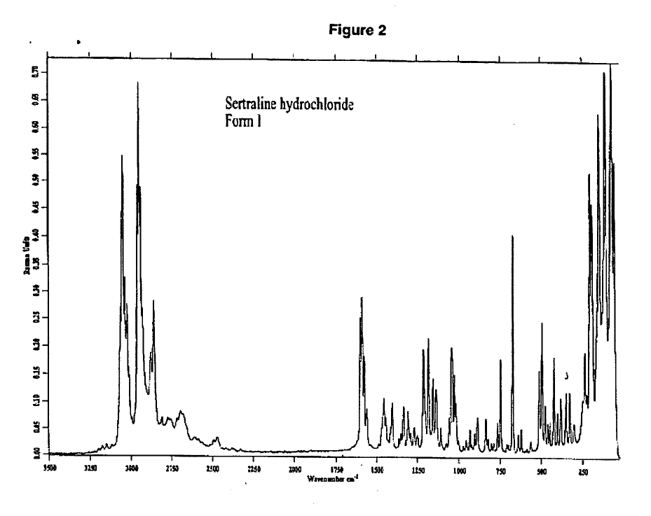

FIG. 3 is a characteristic X-ray powder diffraction pattern for polymorphic form II

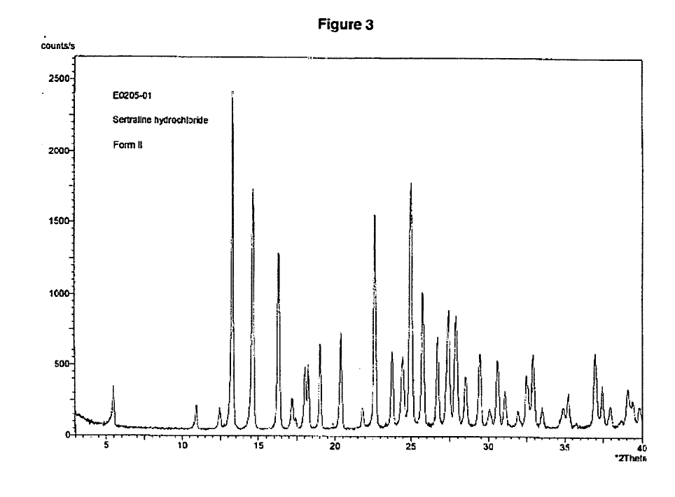

FIG. 4 is a characteristic Raman spectrum of polymorphic form II.

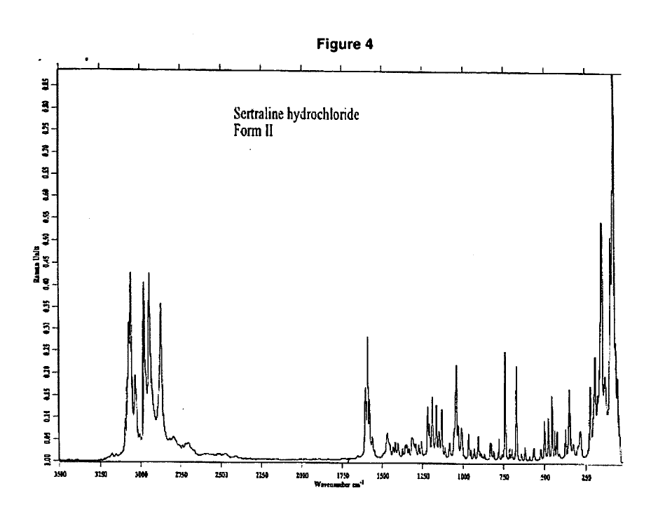

1. Improved process for the preparation of sertraline hydrochloride polymorphic form II by addition of hydrogen chloride to a solution of sertraline free amine in a ketone, characterized in that the solution is heated before addition of the hydrogen chloride, and seeded with some crystals of polymorphic form II.

2. A process according to claim 1 wherein the solution of sertraline free amine is seeded with some crystals of polymorphic form II and subsequently hydrogen chloride is added.

3. A process according to claim 1 wherein hydrogen chloride is added to the solution of sertraline free amine and subsequently the solution is seeded with some crystals of polymorphic form II.

4. A process according to claim 1, wherein a solution of sertraline free amine in a water-immiscible ketone is used.

5. A process according to claim 1, wherein the ketone is of formula R₁—CO—R₂, where R₁ is C₁-C₄alkyl and R₂ is C₂-C₄alkyl.

6. A process according to claim 1, wherein a solution of sertraline free amine in acetone, methyl ethyl ketone, methyl propyl ketone, methyl butyl ketone or methyl isobutyl ketone is used.

7. A process according to claim 1, wherein the amount of seeding crystals used is 1 to 10 mol-%, based on the molar amount of sertraline.

8. A process according to claim 1, wherein gaseous hydrogen chloride is added.

9. A process according to claim 1, wherein the solution is heated to 45-80° C. prior to addition of hydrogen chloride.

10. A process according to claim 1, wherein hydrogen chloride is added until a pH less than 5.5 is reached.

11. A process according to claim 1, wherein sertraline hydrochloride form II is separated from the liquid phase, washed with a pharmaceutically acceptable solvent and dried.

12. A process according to claim 4, wherein the solution of sertraline free amine is obtained after
a) treating a solution of sertraline mandelate in the water immiscible ketone with an aqueous hydroxide,
b) phase separation and
c) washing the ketone solution with water.

13. A pharmaceutical composition comprising an amount of a sertraline hydrochloride form II obtained in the process of claim 1 effective in treating depressions, anxiety-related disorders, obesity, chemical dependencies, or addictions or premature ejaculations in a human, and a pharmaceutically acceptable carrier.
